(19) United States
(12) Patent Application Publication
(10) Pub. No.: US 2017/0325743 A1
(43) Pub. Date: Nov. 16, 2017

(54) REMOTE MONITORING DEVICE

(71) Applicant: Szu Chi LO, Nantou City (TW)

(72) Inventor: Szu Chi LO, Nantou City (TW)

(21) Appl. No.: 15/150,666

(22) Filed: May 10, 2016

(51) Int. Cl. A61B 5/00; A61B 5/0205; G08B 21/18; H04W 4/02; H04B 5/00; A61B 5/11

(52) U.S. Cl. CPC A61B 5/6831; H04W 4/02; H04B 5/0025; A61B 5/02055; G08B 21/18; A61B 5/1112

(57) ABSTRACT

A remote monitoring device is connected to a rubber strap and contains: a controller, a global system for mobile communication (GSM), a global positioning system (GPS), a Wi-Fi & blue tooth, and a near field communication (NFC). The controller includes a microprocessor, a blood pressure sensing unit, a heartbeat sensing unit, a temperature sensing unit, and a wireless signal transmitting unit. The blood pressure sensing unit is configured to sense blood pressure of a care-receiving person, the heartbeat sensing unit is used to sense heart beating of the care-receiving person, the temperature sensing unit is employed to sense a temperature of the care-receiving person, and the microprocessor receives, compares, calculates and sends sensed signals of the blood pressure, the heart beating, and the temperature. The GSM, the GPS and the Wi-Fi & blue tooth are joined with the controller, respectively.

### FIELD OF THE INVENTION

The present invention relates to a remote monitoring device which senses and monitors blood pressure, heart beating, and temperature of a care-receiving person in a remote distance.

### BACKGROUND OF THE INVENTION

A conventional remote monitoring device contains a camera configured to shoot images of a care-receiving person which are sent to a monitor center via a communication network, such that a carer learns whether the care-receiving person is healthful or not. The conventional remote monitoring device also contains a wireless signal transmitter worn or hung on hand or neck of the care-receiving person. Furthermore, a wireless signal receiver is held by the carer so as to receive wireless signals in a set range from the wireless signal transmitter.

However, the remote monitoring device has defects as follows:

(1). The camera cannot shoot the images over the set range.

(2). The carer has to view the images constantly so as to monitor the care-receiving person's condition.

(3). The camera cannot determine whether blood pressure, heart beating, and temperature of the care-receiving person are in an allowable range.

(4). The wireless signal transmitter and the wireless signal receiver cannot send and receive wireless signals in a remote distance, respectively.

(5). The carer has to hold the wireless signal receiver within a limited distance.

(6). The wireless signal transmitter cannot learn the blood pressure, the heart beating, and the temperature of the care-receiving person.

The present invention has arisen to mitigate and/or obviate the afore-described disadvantages.

### SUMMARY OF THE INVENTION

The primary objective of the present invention is to provide a remote monitoring device which senses and monitors blood pressure, heart beating, and temperature of a care-receiving person in a remote distance by using a monitoring receiver in a network transmission manner.

To obtain above-mentioned objective, a remote monitoring device provided by the present invention is connected to a rubber strap and contains: a controller, a global system for mobile communication (GSM), a global positioning system (GPS), and a Wi-Fi & blue tooth.

The controller includes a microprocessor, a blood pressure sensing unit, a heartbeat sensing unit, a temperature sensing unit, and a wireless signal transmitting unit.

The blood pressure sensing unit is configured to sense blood pressure of a care-receiving person, the heartbeat sensing unit is used to sense heart beating of the care-receiving person, the temperature sensing unit is employed to sense a temperature of the care-receiving person, and the microprocessor receives, compares, and calculates if sensed signals of the blood pressure, the heart beating, and the temperature are in an allowable range, wherein the microprocessor sends the sensed signals to the wireless signal transmitting unit after receiving, comparing, and calculating the sensed signals so that the wireless signal transmitting unit converts the sensed signals into wireless signals.

The GSM is in connection with the controller so as to converts the wireless signals into wireless telecommunication signals;

The GPS is coupled with the controller so as to send the wireless signals to a positioning satellite, such that a monitoring receiver learns a position of the care-receiving person.

The Wi-Fi & blue tooth is joined with the controller and converts the sensed signals into wireless network signals which are sent to the monitoring receiver in a network transmission manner.

### BRIEF DESCRIPTION OF THE DRAWINGS

FIG. 1 is a block diagram showing the assembly of a remote monitoring device according to a preferred embodiment of the present invention.

FIG. 2 is a cross sectional view showing the assembly of the remote monitoring device according to the preferred embodiment of the present invention.

FIG. 3 is another cross sectional view showing the assembly of the remote monitoring device according to the preferred embodiment of the present invention.

### DETAILED DESCRIPTION OF THE PREFERRED EMBODIMENTS

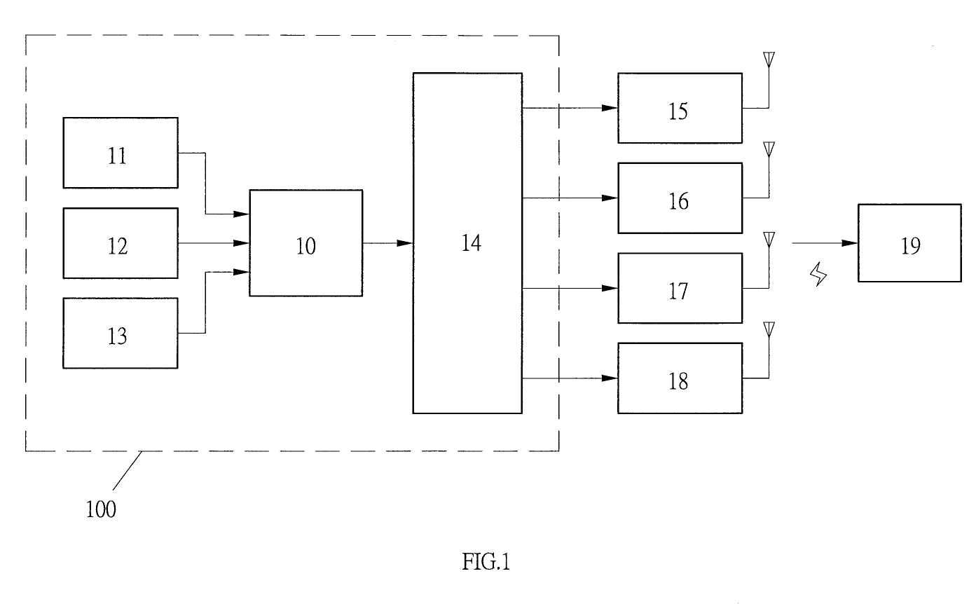

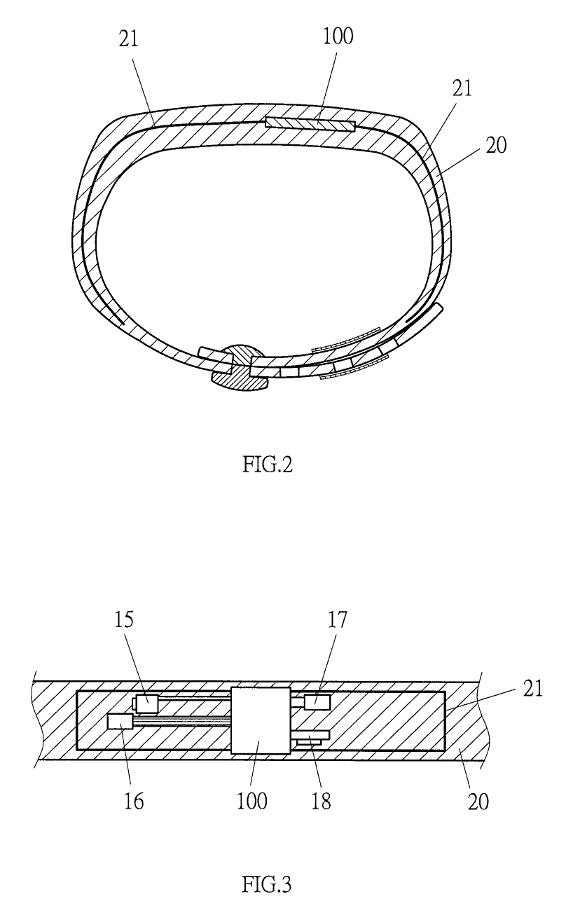

With reference to FIGS. 1-3, a remote monitoring device according to a preferred embodiment of the present invention is connected to a rubber strap 20 and comprises: a controller 100, a global system for mobile communication (GSM) 15, a global positioning system (GPS) 16, a Wi-Fi & blue tooth 17, and a near field communication (NFC) 18.

The controller 100 includes a microprocessor 10, a blood pressure sensing unit 11, a heartbeat sensing unit 12, a temperature sensing unit 13, and a wireless signal transmitting unit 14. The blood pressure sensing unit 11 is configured to sense blood pressure of a care-receiving person, the heartbeat sensing unit 12 is used to sense heart beating of the care-receiving person, the temperature sensing unit 13 is employed to sense a temperature of the care-receiving person, and the microprocessor 10 receives, compares, and calculates if sensed signals of the blood pressure, the heart beating, and the temperature are in an allowable range, wherein the microprocessor 10 sends the sensed signals to the wireless signal transmitting unit 14 after receiving, comparing, and calculating the sensed signals so that the wireless signal transmitting unit 14 converts the sensed signals into wireless signals. The GSM 15 is in connection with the controller 100 so as to converts the wireless signals into wireless telecommunication signals. The GPS 16 is coupled with the controller 100 so as to send the wireless signals to a positioning satellite, such that a monitoring receiver 19 learns a position of the care-receiving person. The Wi-Fi & blue tooth 17 is joined with the controller 100 and converts the sensed signals into wireless network signals which are sent to the monitoring receiver 19 in a network transmission manner. The NFC 18 is in connection with the controller 100 and transmits near field wireless signals to a near field receiver. The rubber strap 20 includes a signal feedback wire 21 connected with the controller 100, such that when the rubber strap 20 is broken to tear off the signal feedback wire 21, the controller 100 informs the monitoring receiver 19 that the rubber strap 20 is broken.

Accordingly, when the care-receiving person wears the rubber strap 20, the blood pressure sensing unit 11 senses the blood pressure, the heart heartbeat sensing unit 12 senses the heart beating, and the temperature sensing unit 13 senses the temperature, thereafter the microprocessor 10 sends the sensed signals to the wireless signal transmitting unit 14, the wireless signal transmitting unit 14 sends the wireless signals to the GSM 15, the GPS 16, and the Wi-Fi & blue tooth 17. In addition, the NFC 18 transmits the near field wireless signals in a near distance so that a carer learns the blood pressure, the heart beating, and the temperature of the care-receiving person by using the monitoring receiver 19. The monitoring receiver 19 is a mobile phone or a computer network, and the positioning satellite acquires the position of the care-receiving person. Preferably, when the rubber strap 20 is broken to tear off the signal feedback wire 21, the controller 100 informs the monitoring receiver 19 that the rubber strap 20 is broken.

Thereby, the remote monitoring device is adapted for signal transmission of internet or telecommunication, such that the carer learns the blood pressure, the heart beating, and the temperature of the care-receiving person in a remote distance by way of satellite communications network so as to call health care unit in emergency.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A remote monitoring device being connected to a rubber strap and comprising: a controller, a global system for mobile communication (GSM), a global positioning system (GPS), and a Wi-Fi & blue tooth;
wherein the controller includes a microprocessor, a blood pressure sensing unit, a heartbeat sensing unit, a temperature sensing unit, and a wireless signal transmitting unit;
wherein the blood pressure sensing unit is configured to sense blood pressure of a care-receiving person, the heartbeat sensing unit is used to sense heart beating of the care-receiving person, the temperature sensing unit is employed to sense a temperature of the care-receiving person, and the microprocessor receives, compares, and calculates if sensed signals of the blood pressure, the heart beating, and the temperature are in an allowable range, wherein the microprocessor sends the sensed signals to the wireless signal transmitting unit after receiving, comparing, and calculating the sensed signals so that the wireless signal transmitting unit converts the sensed signals into wireless signals;
wherein the GSM is in connection with the controller so as to converts the wireless signals into wireless telecommunication signals;
wherein the GPS is coupled with the controller so as to send the wireless signals to a positioning satellite, such that a monitoring receiver learns a position of the care-receiving person; and
wherein the Wi-Fi & blue tooth is joined with the controller and converts the sensed signals into wireless network signals which are sent to the monitoring receiver in a network transmission manner.

2. The remote monitoring device as claimed in claim 1, wherein a near field communication (NFC) is in connection with the controller.

3. The remote monitoring device as claimed in claim 1, wherein the rubber strap includes a signal feedback wire connected with the controller, such that when the rubber strap is broken to tear off the signal feedback wire, the controller informs the monitoring receiver that the rubber strap is broken.
